(19) United States
(12) Patent Application Publication
(10) Pub. No.: US 2007/0288038 A1
(43) Pub. Date: Dec. 13, 2007

(54) Medical Retrieval Devices and Methods

(76) Inventor: Frank Bimbo, Lawrenceville, GA (US)

Correspondence Address: BRIAN M. BURN, ESQ.;C.R. BARD UROLOGICAL DIVISION, P.O. BOX 52050, c/o PORTFOLIOIP, MINNEAPOLIS, MN, 55402, US

(21) Appl. No.: 11/761,721

(22) Filed: Jun. 12, 2007

Related U.S. Application Data
(60) Provisional application No. 60/804,577, filed on Jun. 13, 2006.

(51) Int. Cl. A61B 17/22

(52) U.S. Cl. 606/127

(57) ABSTRACT

A medical retrieval device may include a first portion having proximal and distal ends, a second portion having proximal and distal ends, and a grasping portion. The second portion may surround at least a portion of the first portion. The grasping portion may be movable between retracted and extended configurations. The grasping portion may include a proximal end, a free distal end, and a plurality of grasping members. Each grasping member may include a plurality of grasping elements extending between the proximal and free distal ends of the grasping portion. At least one of the grasping elements may be connected to the distal end of the first portion and extend to the free end, and at least one other grasping element may be connected to the distal end of the second portion and extend to the free end. Relative movement between the first and second portions may cause the grasping portion to move between the retracted and extended configurations.

The present application claims priority to U.S. Provisional Application No. 60/804,577, filed Jun. 13, 2006, the disclosure of which is incorporated herein by reference in its entirety.

### TECHNICAL FIELD

The present invention is directed to medical retrieval devices. More particularly, the present invention is directed to medical retrieval devices and methods including a grasping portion for grasping a target object in a body lumen.

### BACKGROUND

Some conventional medical baskets and graspers include a sheath with a stone basket or grasping prongs inside of the sheath. The basket or prongs are extended from and withdrawn into the sheath to open and close the device when retrieving an object, such as a kidney stone, from a body lumen. Since the basket or prongs are withdrawn into the sheath for insertion into and removal from the body lumen, the retracted configuration is limited to the inside diameter of the sheath. In addition, the devices are limited by the amount of material that will fit into the sheath. Further, devices being withdrawn into a sheath tend to withdraw away from the target object.

It may be desirable to provide a retrieval device that does not need to be withdrawn into a sheath in order to maximize the material area available to design the grasping device. Such a device may also better close around a target object rather than withdrawing away from it.

Some conventional retrieval devices are constructed with joints that are prone to breakage. It may be desirable to construct a medical retrieval device from a single piece of unitary construction so as to eliminate, or at least substantially eliminate, breakable joints.

The medical retrieval devices and methods of the present invention may solve one or more of the problems set forth above.

### SUMMARY OF THE INVENTION

According to various aspects of the disclosure, a medical retrieval device may include a first portion having proximal and distal ends, a second portion having proximal and distal ends, and a grasping portion. The second portion may surround at least a portion of the first portion. The grasping portion may be movable between retracted and extended configurations. The grasping portion may include a proximal end, a free distal end, and a plurality of grasping members. Each grasping member may include a plurality of grasping elements extending between the proximal and free distal ends of the grasping portion. At least one of the grasping elements may be connected to the distal end of the first portion and extend to the free end, and at least one other grasping element may be connected to the distal end of the second portion and extend to the free end. Relative movement between the first and second portions may cause the grasping portion to move between the retracted and extended configurations.

In some aspects of the disclosure, a medical retrieval device may include a first portion having an inside diameter, a proximal end, and a distal end, and a second portion surrounding at least a portion of the first portion. The second portion may include a grasping portion movable between a retracted configuration and an extended configuration, the grasping portion may be external to the inside diameter of the first portion in the retracted and extended configurations. The grasping portion may extend distally from the distal end of the first portion in the retracted and extended configurations and terminate in a free distal end. Relative movement between the first and second portions may cause the grasping portion to move between the retracted and extended configurations.

In accordance with various aspects of the disclosure, an exemplary medical retrieval device may include a first portion having an inside diameter, a proximal end, and a distal end, and a second portion surrounding at least a portion of the first portion. The second portion may include a grasping portion movable between a retracted configuration and an extended configuration. The grasping portion may extend distally from the distal end of the second portion in the retracted and extended configurations and terminate in a free distal end. Relative movement between the first and second portions may cause the grasping portion to move between the retracted and extended configurations without relative translational motion between the free distal end and the first and second portions.

### BRIEF DESCRIPTION OF THE DRAWINGS

FIG. 1 is a front perspective view of a medical retrieval device in a first configuration in accordance with exemplary aspects of the present invention;

FIG. 2 is a front perspective view of the medical retrieval device of FIG. 1 in a second configuration;

FIG. 3 is a front plan view of a medical retrieval device in accordance with exemplary aspects of the present invention;

FIG. 4 is a top plan view of the medical retrieval device of FIG. 3;

FIG. 5 is a front perspective view of a medical retrieval device in a first configuration in accordance with exemplary aspects of the present invention;

FIG. 6 is a front perspective view of the medical retrieval device of FIG. 5 in a second configuration;

FIG. 7 is a front perspective view of the medical retrieval device of FIG. 1 with an alternative grasping portion;

FIG. 8 is a front perspective view of the medical retrieval device of FIG. 1 with an alternative grasping portion;

FIG. 9 is a front perspective view of the medical retrieval device of FIG. 1 with an alternative grasping portion; and

FIG. 10 is a front perspective view of the medical retrieval device of FIG. 1 with an alternative grasping portion.

### DESCRIPTION

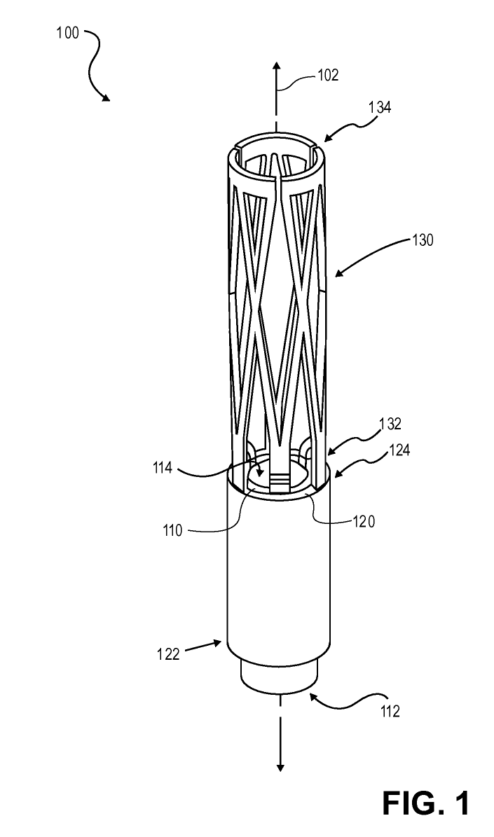

An exemplary embodiment of a medical retrieval device 100 is illustrated in FIG. 1. The medical retrieval device 100 may include a first portion 110 and a second portion 120 aligned along a longitudinal axis 102 of the device 100. The first portion 110 may comprise, for example, a push rod or a hypo tube defining at least one lumen, and the second portion 120 may comprise, for example, a sheath. The second portion 120 may surround at least a portion of the first portion 110, and the first and second portions 110, 120 may be slidable and/or rotatable relative to one another.

The first portion 110 may comprise a proximal end 112 and a distal end 114, and the second portion 120 may comprise a proximal end 122 and a distal end 124. A grasping portion 130 may extend from the distal ends 114, 124 of the first and second portion 110, 120, respectively, and may include a proximal end 132 and a free distal end 134. According to various aspects, the first, second, and grasping portions 110, 120, 130 may comprise a single piece of unitary construction, thus eliminating joints associated with connection regions.

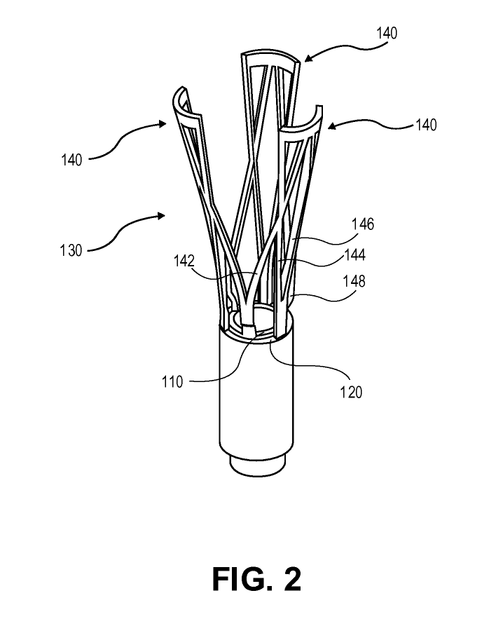

Referring to FIG. 2, the grasping portion 130 may comprise a plurality of grasping elements 142, 144, 146, 148 defining a plurality of grasping members 140. For clarity, the grasping elements 142, 144, 146, 148 for only one grasping member 140 are numbered. The grasping members 140 may be operably connected to one another in a joint-free manner, for example, via a single piece of unitary construction. According to various aspects, the grasping portion 130 may comprise, for example, three grasping members 140. It should be appreciated that in various aspects the grasping portion 130 may comprise two grasping members or more than three grasping members.

According to various aspects, the grasping members 140 may be cut from a solid member, for example, a solid tube. In some exemplary aspects, the solid member may comprise, for example, a nitinol tubing or another shape memory alloy tubing that is etched, for example, via laser etching, to a desired configuration or pattern. The tubing may have a circular cross-section, or any other desired shape. According to some aspects, the grasping members may comprise a weave of metal mesh in a desired configuration. The first portion 110 and/or the second portion 120 may be constructed of stainless steel or a shape memory alloy such as, for example, nitinol.

Each grasping member 140 may comprise a plurality of interconnected grasping elements 142, 144, 146, 148. For example, as shown in FIGS. 1 and 2, each grasping member 140 may comprise four interconnected grasping elements 142, 144, 146, 148. The grasping elements 142, 144, 146, 148 may be interconnected to one another in a joint-free manner, for example, via a single piece of unitary construction.

At least one of the grasping elements 142, 148 of each grasping member 140 may extend from a distal end 114 of the first portion 110 and at least one of the grasping elements 144, 146 of each grasping member 140 may extend from a distal end 124 of the second portion 120. Two or more of the grasping elements 142, 144, 146, 148 of each grasping member 140 may be connected to one another in a joint-free manner, for example, via a single piece of unitary construction, at the free distal end 134 of the grasping portion 130.

As shown in FIG. 1, in a retracted configuration, the grasping portion 130 may have a diameter substantially the same as the diameter of the second portion 120, except for portions of the grasping elements 142, 148 extending from the distal end 114 of the first portion 110. Substantially the entire grasping portion 130 may be external to the inside dimension, for example, the diameter, of the first portion 110.

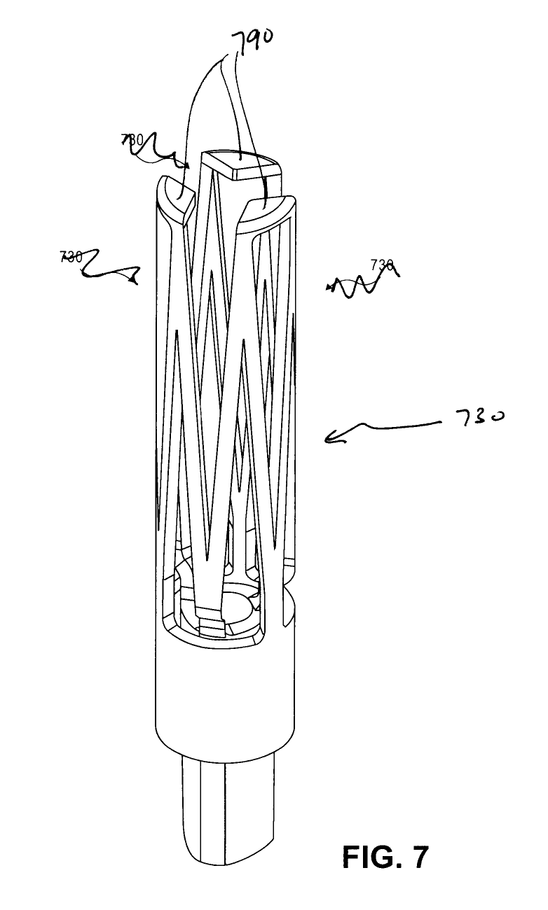

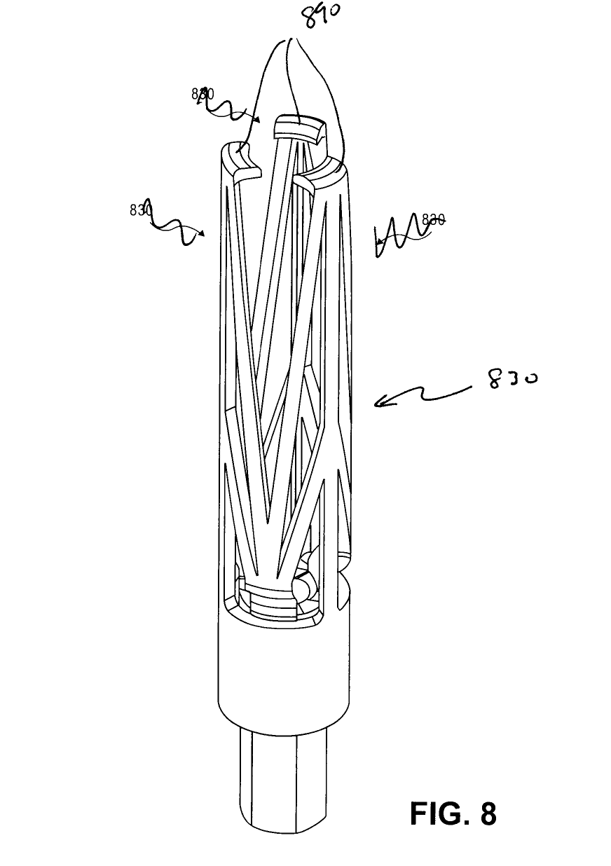

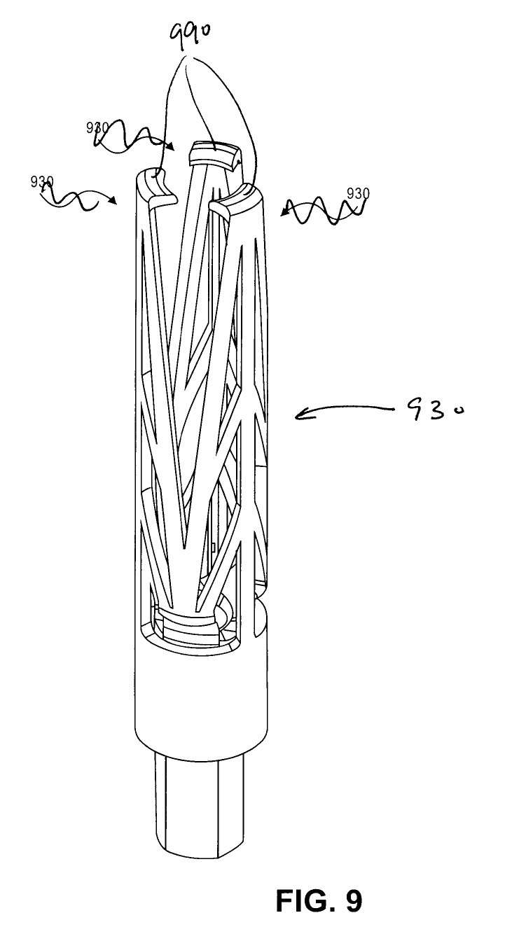

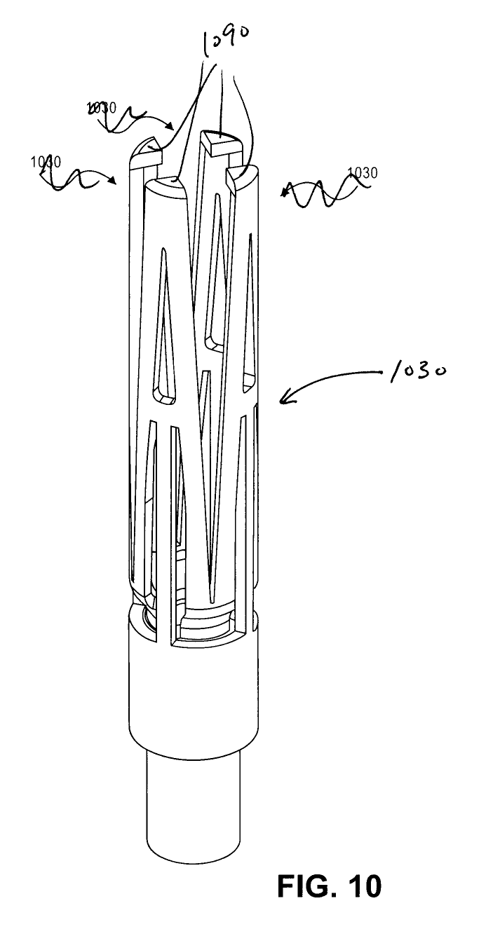

Alternative configurations for the grasping portion 130 are illustrated in FIGS. 7-10 as grasping portions 730, 830, 930, and 1030. As shown in FIGS. 7-10, each grasping portion 140 may include two or more gripping portions 790, 890, 990, 1090 at the distal end of the respective grasping portion 730, 830, 930, 1030. The gripping portions 790, 890, 990, 1090 may extend inward toward a center region of the retrieval device so as to facilitate retrieval of an object, for example, a kidney stone.

In operation, the first portion 110 may be urged to move relative to the second portion 120 via selective operation of an actuating member (not shown) in order to move the grasping portion 130 between retracted and extended configurations. For example, in some aspects, the grasping portion 130 may be moved from a retracted configuration (FIG. 1) to an extended configuration (FIG. 2) via relative translational movement between the first and second portions 110, 120 along the axis 102. As shown in FIG. 2, the distal end 114 of the first portion 110 is moved past the distal end 124 of the second portion 120 to achieve the extended configuration.

In the extended configuration, the grasping members 140 extend outward from the outer diameter of the second portion 120. Despite the relative translational movement between the first and second portions 110, 120, the distal end 134 of the grasping portion 130 does not translate relative to the distal end 124 of the second portion 120. However, the movement of the grasping portion 130 between the retracted and extending configurations does include a minimal component along the longitudinal axis 102 in addition to the movement of the grasping portion 130 in a direction transverse to the longitudinal axis 102.

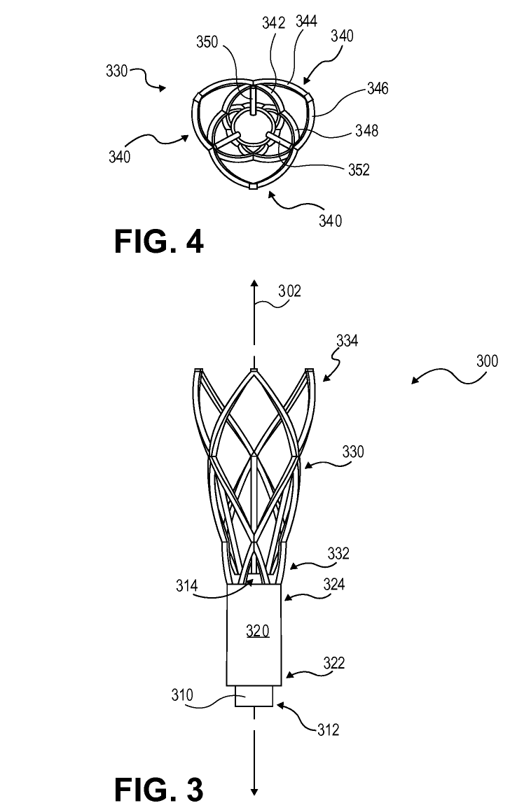

Another exemplary embodiment of a medical retrieval device 300 is illustrated in FIGS. 3 and 4. The medical retrieval device 300 may include a first portion 310 and a second portion 320 aligned along a longitudinal axis 302 of the device 300. The first portion 310 may comprise, for example, a push rod or a hypo tube defining at least one lumen, and the second portion 320 may comprise, for example, a sheath. The second portion 320 may surround at least a portion of the first portion 310, and the first and second portions 310, 320 may be slidable and/or rotatable relative to one another

The first portion 310 may comprise a proximal end 312 and a distal end 314, and the second portion 320 may comprise a proximal end 322 and a distal end 324. A grasping portion 330 may extend from the distal ends 314, 324 of the first and second portions 310, 320, respectively, and may include a proximal end 332 and a free distal end 334. According to various aspects, the first, second, and grasping portions 310, 320, 330 may comprise a single piece of unitary construction, thus eliminating joints associated with connection regions.

Referring to FIG. 4, the grasping portion 330 may comprise a plurality of grasping elements 342, 344, 346, 348, 350, 352 defining a plurality of grasping members 340. For clarity, the grasping elements 342, 344, 346, 348, 350, 352 for only one grasping member 340 are numbered. The grasping members 340 may be operably connected to one another in a joint-free manner, for example, via a single piece of unitary construction. According to various aspects, the grasping portion 330 may comprise, for example, three grasping members 340. It should be appreciated that in various aspects the grasping portion 330 may comprise two grasping members or more than three grasping members

According to various aspects, the grasping members 340 may be cut from a solid member, for example, a solid tube. In some exemplary aspects, the solid member may comprise, for example, a nitinol tubing or another shape memory alloy tubing that is etched, for example, via laser etching, to a desired configuration or pattern. The tubing may have a circular cross-section, or any other desired shape. According to some aspects, the grasping members may comprise a weave of metal mesh in a desired configuration. The first portion 310 and/or the second portion 320 may be constructed of stainless steel or a shape memory alloy such as, for example, nitinol.

Each grasping member 340 may comprise a plurality of interconnected grasping elements 342, 344, 346, 348, 350, 352. For example, as shown in FIGS. 3 and 4, each grasping member 140 may comprise six interconnected grasping elements 342, 344, 346, 348, 350, 352. The grasping elements 342, 344, 346, 348, 350, 352 may be interconnected to one another in a joint-free manner, for example, via a single piece of unitary construction. Some of the grasping elements 342, 344, 346, 348, 350, 352 may cooperate to define more than one grasping member 340.

At least one of the grasping elements 350, 352 of each grasping member 340 may extend from a distal end 314 of the first portion 310 and at least one of the grasping elements 342, 344, 346, 348 of each grasping member 340 may extend from a distal end 324 of the second portion 320. Two or more of the grasping elements 342, 344, 346, 348, 350, 352 of each grasping member 340 may be connected to one another in a joint-free manner, for example, via a single piece of unitary construction, at the free distal end 334 of the grasping portion 330

In a retracted configuration (not shown), the grasping portion 330 may have a diameter substantially the same as the diameter of the second portion 320, except for portions of the grasping elements 350, 352 extending from the distal end 314 of the first portion 310. Substantially the entire grasping portion 330 may be external to the inside dimension, for example, the diameter, of the first portion 310

In operation, the first portion 310 may be urged to move relative to the second portion 320 via selective operation of an actuating member (not shown) in order to move the grasping portion 330 between retracted and extended configurations. For example, in some aspects, the grasping portion 330 may be moved from a retracted configuration to an extended configuration (FIGS. 3 and 4) via relative translational movement between the first and second portions 310, 320 along the axis 302. As shown in FIG. 3, the distal end 314 of the first portion 310 is moved past the distal end 324 of the second portion 320 to achieve the extended configuration.

In the extended configuration, the grasping members 340 extend outward from the outer diameter of the second portion 320. Despite the relative translational movement between the first and second portions 310, 320, the distal end 334 of the grasping portion 330 does not translate relative to the distal end 324 of the second portion 320. However, the movement of the grasping portion 330 between the retracted and extending configurations does include a minimal component along the longitudinal axis 302 in addition to the movement of the grasping portion 330 in a direction transverse to the longitudinal axis 302.

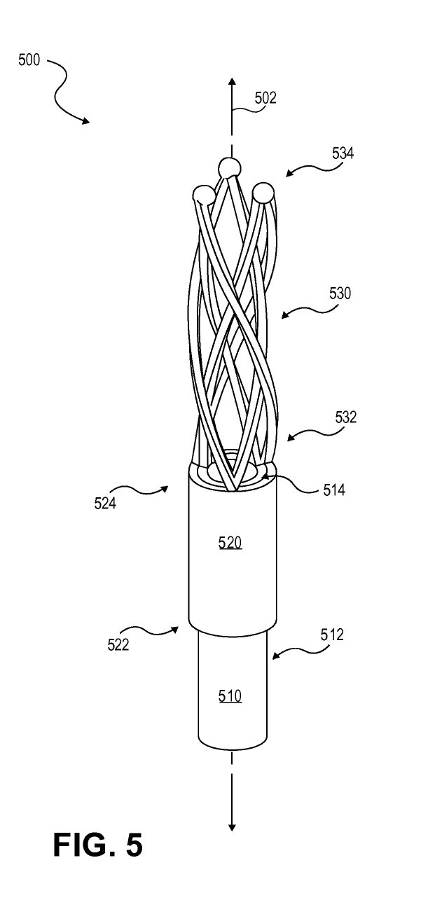

An exemplary embodiment of a medical retrieval device 500 is illustrated in FIG. 5. The medical retrieval device 500 may include a first portion 510 and a second portion 520 aligned along a longitudinal axis 502 of the device 500. The first portion 510 may comprise, for example, a push rod or a hypo tube defining at least one lumen, and the second portion 520 may comprise, for example, a sheath. The second portion 520 may surround at least a portion of the first portion 510, and the first and second portions 510, 520 may be slidable and/or rotatable relative to one another

The first portion 510 may comprise a proximal end 512 and a distal end 514, and the second portion 520 may comprise a proximal end 522 and a distal end 524. A grasping portion 530 may extend from the distal ends 514, 524 of the first and second portions 510, 520, respectively, and may include a proximal end 532 and a free distal end 534. According to various aspects, the first, second, and grasping portions 510, 520, 530 may comprise a single piece of unitary construction, thus eliminating joints associated with connection regions.

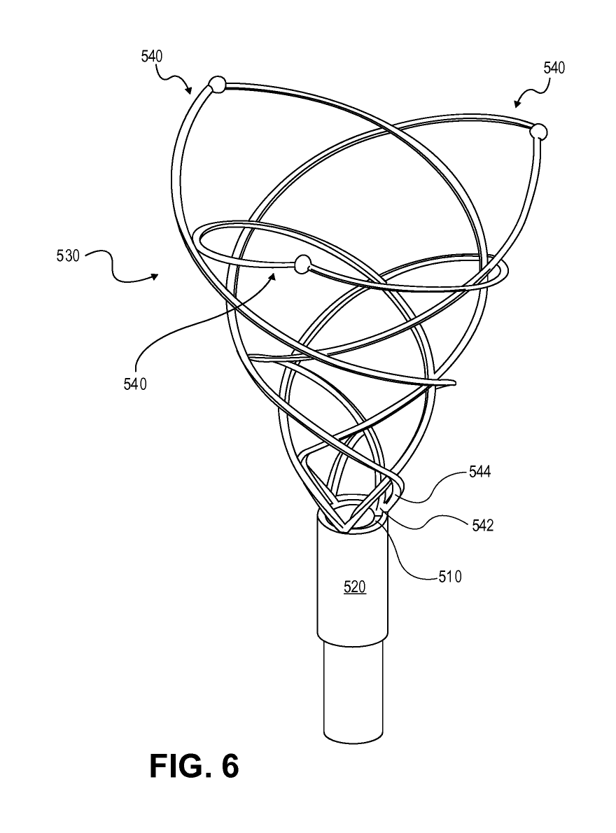

Referring to FIG. 6, the grasping portion 530 may comprise a pair of grasping elements 542, 544 defining a plurality of grasping members 540. For clarity, the grasping elements 542, 544 for only one grasping member 540 are numbered. The grasping members 540 may be operably connected to one another in a joint-free manner, for example, via a single piece of unitary construction. According to various aspects, the grasping portion 530 may comprise, for example, three grasping members 540. It should be appreciated that in various aspects the grasping portion 530 may comprise two grasping members or more than three grasping members.

According to various aspects, the grasping members 540 may be cut from a solid member, for example, a solid tube. In some exemplary aspects, the solid member may comprise, for example, a nitinol tubing or another shape memory alloy tubing that is etched, for example, via laser etching, to a desired configuration or pattern. The tubing may have a circular cross-section, or any other desired shape. According to some aspects, the grasping members may comprise a weave of metal mesh in a desired configuration. The first portion 510 and/or the second portion 520 may be constructed of stainless steel or a shape memory alloy such as, for example, nitinol.

Each grasping member 540 may comprise, for example, a pair of interconnected grasping elements 542, 544. The grasping elements 542, 544 may be interconnected to one another in a joint-free manner, for example, via a single piece of unitary construction.

As shown in FIG. 6, one of the grasping elements 542 of each grasping member 540 may extend from the distal end 514 of the first portion 510 and one of the grasping elements 544 of each grasping member 540 may extend from the distal end 524 of the second portion 520. Each pair of the grasping elements 542, 544 of each grasping member 540 may be connected to one another in a joint-free manner, for example, via a single piece of unitary construction, at the free distal end 534 of the grasping portion 530.

As shown in FIG. 5, in a retracted configuration, the grasping portion 530 may have a diameter substantially the same as the diameter of the second portion 520, except for portions of the grasping elements 542 extending from the distal end 514 of the first portion 510. Substantially the entire grasping portion 530 may be external to the inside dimension, for example, the diameter, of the first portion 510.

In operation, the first portion 510 may be urged to move relative to the second portion 520 via selective operation of an actuating member (not shown) in order to move the grasping portion 530 between retracted and extended configurations. For example, in some aspects, the grasping portion 530 may be moved from a retracted configuration (FIG. 5) to an extended configuration (FIG. 6) via relative rotational movement between the first and second portions 510, 520 about the axis 602. As shown in FIG. 6, the distal end 514 of the first portion 510 is not moved past the distal end 524 of the second portion 520 to achieve the extended configuration.

In the extended configuration, the grasping members 540 extend outward from the outer diameter of the second portion 520. With the relative rotational movement between the first and second portions 510, 520, the distal end 534 of the grasping portion 530 does not translate relative to the distal end 524 of the second portion 520. However, the movement of the grasping portion 530 between the retracted and extended configurations does include a minimal component along the longitudinal axis 502 in addition to the movement of the grasping portion 530 in a direction transverse to the longitudinal axis 502.

In accordance with various aspects of the invention, any of the medical devices 100, 300, 500 disclosed herein may be used to retrieve an object, such as, for example, a kidney stone, from a human body. The medical device 100, 300, 500 may be inserted into a body lumen to a location proximal a target object such as, for example, a kidney stone. Once in the appropriate location, the first portion 110, 310, 510 may be moved relative to the second portion 120, 320, 520 so as to urge the grasping portion 130, 330, 530 from a retracted configuration to an extended configuration. The relative movement may be relative translational and/or rotational movement.

Once moved into an extended configuration, the medical device 100, 300, 500 may be moved to a position such that the grasping portion 130, 330, 530 surrounds at least a portion of the target object. While surrounding at least a portion of the target object, the first portion 110, 310, 510 may be moved relative to the second portion 120, 320, 520 so as to urge the grasping portion 130, 330, 530 from the extended configuration to the retracted configuration to grasp the target object. After the target object is grasped, the medical device 100, 300, 500 may be removed from the body lumen.

It should be appreciated that a laser fiber or other conventional device may be inserted through the first portion 110, 310, 510 and operated to break up the object, for example, a kidney stone, prior to grasping and removal. It should also be appreciated that the grasping portion 130, 330, 530 may grasp and capture a plurality of target objects before being removed from the body lumen.

It will be apparent to those skilled in the art that various modifications and variations can be made in the medical retrieval devices and methods of the present invention without departing from the scope of the invention. Other embodiments of the invention will be apparent to those skilled in the art from consideration of the specification and practice of the invention disclosed herein. It is intended that the specification and examples be considered as exemplary only.

What is claimed is:

1. A medical retrieval device, comprising:
a first portion having a proximal end and a distal end;
a second portion surrounding at least a portion of the first portion, the second portion having a proximal end and a distal end;
a grasping portion movable between a retracted configuration and an extended configuration, the grasping portion comprising
a proximal end,
a free distal end, and
a plurality of grasping members, each grasping member comprising a plurality of grasping elements extending between said proximal and free distal ends of the grasping portion, at least one of said grasping elements being integral with said distal end of the first portion and extending to said free end, and at least one other of said grasping elements being connected to said distal end of the second portion and extending to said free end,
wherein relative movement between the first and second portions causes the grasping portion to move between said retracted and extended configurations.

2. The device of claim 1, wherein the second portion comprises a sheath.

3. The device of claim 2, wherein the first portion defines at least one lumen.

4. The device of claim 1, wherein relative rotational movement between the first and second portions causes the grasping portion to move between said retracted and extended configurations.

5. The device of claim 1, wherein relative translational movement between the first and second portions causes the grasping portion to move between said retracted and extended configurations.

6. The device of claim 1, wherein the grasping portion is external to the inside diameter of the first portion in the retracted and extended configurations.

7. The device of claim 1, wherein said relative movement between the first and second portions causes the grasping portion to move between said retracted and extended configurations without relative translational motion between said free distal end and the first and second portions.

8. A medical retrieval device, comprising:
a first portion having an inside diameter, a proximal end, and a distal end; and
a second portion surrounding at least a portion of the first portion, the second portion comprising a grasping portion movable between a retracted configuration and an extended configuration, the grasping portion being external to the inside diameter of the first portion in the retracted and extended configurations, the grasping portion extending distally from the distal end of the first portion in said retracted and extended configurations and terminating in a free distal end,
wherein relative movement between the first and second portions causes the grasping portion to move between said retracted and extended configurations.

9. The device of claim 8, wherein the second portion comprises a sheath.

10. The device of claim 9, wherein the first portion defines at least one lumen.

11. The device of claim 10, wherein relative rotational movement between the first and second portions causes the grasping portion to move between said retracted and extended configurations.

12. The device of claim 10, wherein relative translational movement between the first and second portions causes the grasping portion to move between said retracted and extended configurations.

13. The device of claim 10, wherein the grasping portion comprises:
a proximal end;
a free distal end; and
a plurality of grasping members, each grasping member comprising a plurality of grasping elements extending between said proximal and free distal ends of the grasping portion, at least one of said grasping elements being connected to said distal end of the first portion and extending to said free end, and at least one other of said grasping elements being connected to said distal end of the second portion and extending to said free end.

14. The device of claim 10, wherein said relative movement between the sheath and the outer member causes the grasping portion to move between said retracted and extended configurations without relative translational motion between said free distal end and the first and second portions.

15. A medical retrieval device, comprising:
a first portion having an inside diameter, a proximal end, and a distal end; and
a second portion surrounding at least a portion of the first portion, the second portion comprising a grasping portion movable between a retracted configuration and an extended configuration, the grasping portion extending distally from the distal end of the second portion in said retracted and extended configurations and terminating in a free distal end,
wherein relative movement between the first and second portions causes the grasping portion to move between said retracted and extended configurations without relative translational motion between said free distal end and the first and second portions.

16. The device of claim 15, wherein the second portion comprises a sheath.

17. The device of claim 16, wherein the first portion defines at least one lumen.

18. The device of claim 15, wherein relative rotational movement between the first and second portions causes the grasping portion to move between said retracted and extended configurations.

19. The device of claim 15, wherein relative translational movement between the first and second portions causes the grasping portion to move between said retracted and extended configurations.

20. The device of claim 15, wherein the grasping portion is external to the inside diameter of the first portion in the retracted and extended configurations.
